(19) United States
(12) Patent Application Publication
(10) Pub. No.: US 2024/0238453 A1
(43) Pub. Date: Jul. 18, 2024

(54) COMPLEX FOR BIOIMAGING, AND DIAGNOSIS OR TREATMENT OF CANCER

(71) Applicant: KOREA RESEARCH INSTITUTE OF BIOSCIENCE AND BIOTECHNOLOGY, Daejeon (KR)

(72) Inventors: Chang-Soo LEE, Daejeon (KR); Kyung Kwan LEE, Daejeon (KR); Jong-Won KIM, Daejeon (KR)

(73) Assignee: KOREA RESEARCH INSTITUTE OF BIOSCIENCE AND BIOTECHNOLOGY, Daejeon (KR)

(21) Appl. No.: 18/289,333

(22) PCT Filed: May 2, 2022
(86) PCT No.: PCT/KR2022/006276

§ 371 Date: Nov. 2, 2023

(30) Foreign Application Priority Data
May 3, 2021 (KR) 10-2021-0057519

(51) Int. Cl. A61K 49/00; A61K 41/00; A61P 35/00; B82Y 5/00; B82Y 20/00

(52) U.S. Cl. CPC A61K 49/0067; A61K 41/0052; A61K 49/005; A61P 35/00; B82Y 5/00; B82Y 20/00

(57) ABSTRACT

The present invention relates to a complex for bioimaging, diagnosis, and treatment of cancer cells. The complex of the present invention comprises fluorescent nanoparticles and a manganese salt conjugated to the surface of the fluorescent nanoparticles, and the fluorescence of the fluorescent nanoparticles in the complex is quenched due to the conjugation of the manganese salt.

### TECHNICAL FIELD

The present invention relates to a complex for bioimaging or for a diagnosis or treatment of a cancer.

### BACKGROUND ART

“Theragnosis” technology is a futuristic diagnostic and therapeutic technology that performs early diagnosis and effective treatment of intractable diseases, including cancers, at the same time and is capable of implementing personalized treatment for patients. In particular, multidisciplinary convergence research in the fields of biomedical engineering and medical and pharmaceutical sciences is essential.

Recently, domestic research has been actively conducted to discover bio-nano source materials necessary for the development of convergence and fusion technologies of ‘Molecular imaging’ and ‘Nanomedicine’ technologies, and to develop innovative diagnostic and therapeutic theragnosis technologies to secure future medical source technologies that can achieve global competitiveness.

As part of this effort, studies have recently been conducted to develop multifunctional nanomaterials that can synergistically perform bioimaging, cancer cell detection, and photothermal effects. Studies are being conducted on various nanoparticles, such as gold nanoparticles, iron oxide nanoparticles, semiconductor quantum dots, polymer nanoparticles, carbon nanoparticles, and graphene, all of which are candidate materials that are capable of integrating and exhibiting various functions.

In particular, carbon fluorescence nanoparticles can be implemented in sizes as small as 10 nm and exhibit good biocompatibility, low or no cytotoxicity, high yield, low cost, and non-blinking characteristics, thus becoming an emerging material in the field of nanomedicine. However, it has been reported that the carbon fluorescence nanoparticles still need to be further studied in terms of emission tunability.

In addition, this is confirmed by studies on photothermal therapy, which can be used to treat cancer at the time of diagnosis, actively being conducted in the field of cancer treatment. The photothermal therapy is a diagnostic and treatment procedure in which light is illuminated on a contrast agent for the diagnosis of a cancer cell to induce heat at the localized position where the cancer cell is present, thereby burning away a solid cancer.

While studies on a method of simultaneously diagnosing and treating cancer have been carried out steadily to date, the method has not been widely used due to a lack of efficiency and unproven safety in the body.

Therefore, there has been an ongoing effort to develop the contrast agent that enables simultaneous diagnosis and treatment of cancer, has high diagnostic and treatment efficiency, and ensures stability in the body.

### DISCLOSURE

### Technical Problem

The present invention is directed to providing a complex that can be used as a contrast agent and a method of manufacturing the same.

In addition, the present invention is directed to providing a composition for bioimaging that includes the complex.

In addition, the present invention is directed to providing a complex capable of targeting a cancer cell to diagnose or treat the cancer, and a method of manufacturing the same.

### Technical Solution

To achieve the objects as described above,

In one aspect of the present invention, there is provided a complex including: a fluorescent nanoparticle; and a manganese salt conjugated to a surface of the fluorescent nanoparticle, in which fluorescence of the fluorescent nanoparticle is in a state of being quenched due to the conjugation of the manganese salt.

In another aspect of the present invention, there is provided a composition for bioimaging including the complex according to the present invention.

In still another aspect of the present invention, there is provided a composition for diagnosis of cancer including the complex according to the present invention.

In yet another aspect of the present invention, there is provided a composition for treatment of cancer including the complex according to the present invention.

### Advantageous Effects

There is an effect of enabling the imaging diagnosis of cancer using the complex of the present invention. In addition, the complex of the present invention has an effect of improving the efficiency of photothermal therapy for cancer cells simultaneously with the imaging diagnosis of cancer.

Specifically, since the fluorescent nanoparticles are quenched due to the conjugation of manganese salt, and then the manganese salt is ionized specifically in the environment in which the cancer cell is present, and the fluorescence of the fluorescent nanoparticles is recovered, the complex of the present invention has an effect of enabling multiplexed bioimaging of the cancer cell through fluorescence imaging and MRI by targeting the cancer cell.

Further, since the complex of the present invention has an effect of absorbing light and dissipating heat, after the complex of the present invention is delivered to the cancer cell, a cancer lesion is illuminated with light and photothermal heat is generated. Therefore, the complex of the present invention has an effect of inhibiting the growth of the cancer cell or further killing the cancer cell.

Furthermore, since the complex of the present invention has excellent thermal stability, there is an effect of enabling stable photothermal treatment even in heat-exposed environments.

### DESCRIPTION OF DRAWINGS

FIG. 1 is a schematic view of a cancer cell treatment mechanism by bioimaging and photothermal therapy of a complex of the present invention.

FIG. 2 is a schematic view of a method of manufacturing the complex according to an embodiment of the present invention.

FIG. 3 is structural analysis results of the complex (MnCO₃@SFPNPs) through various analytical methods according to an embodiment of the present invention. (a) DLS analysis, (b) TEM image, (c) high magnification TEM image, (d) XRD spectrum, (e) FT-IR spectrum, (f) XPS survey spectrum

FIG. 4 is X-ray photoelectron spectroscopy (XPS) analysis results of the complex (MnCO₃@SFPNPs) according to an embodiment of the present invention. Results of polydopamine (PDA), fluorescence polydopamine nanoparticles (SFPNPs) and manganese-fluorescence polydopamine nanoparticle intermediate (Mn@SFPNPs) as precursors and precursors for synthesis are illustrated together.

FIG. 5 is a TEM image of the complex (MnCO₃@SFPNPs) according to an embodiment of the present invention. Results of fluorescence polydopamine nanoparticles (SFPNPs) and manganese-fluorescence polydopamine nanoparticle intermediate (Mn@SFPNPs) are illustrated together.

FIG. 6 is mapping analysis results using TEM analysis of the complex (MnCO₃@SFPNPs) according to an embodiment of the present invention.

FIG. 7 is measurement result graphs of excitation-emission fluorescence spectra (left), quantum efficiency (middle), and fluorescence lifetime (right) of fluorescent polydopamine nanoparticles (SFPNPs).

FIG. 8 is a measurement result graph of fluorescence wavelength and pH stability of fluorescent polydopamine nanoparticles (SFPNPs).

FIG. 9 is result graphs of confirming a fluorescence quenching effect of fluorescent polydopamine nanoparticles (SFPNPs) depending on concentration of manganese metal ions.

FIG. 10 is a result graph of confirming fluorescence quenching reactivity of fluorescent polydopamine nanoparticles (SFPNPs) to heavy metals.

FIG. 11 is result graphs of measuring manganese ion release capacity depending on pH of the complex according to an embodiment of the present invention and changes in magnetic properties depending on manganese ion release.

FIG. 12 is phantom images of the complex depending on time and pH, according to an embodiment of the present invention.

FIG. 13 is a result graph (left) and image (right) of confirming fluorescence recovery characteristics depending on pH of the complex according to an embodiment of the present invention under an acidic condition.

FIG. 14A is an experimental condition for a heat dissipation experiment according to an embodiment of the present invention, and FIG. 14B is a temperature confirmation result using a thermal imaging camera.

FIG. 15A to FIG. 15C illustrates results of heat dissipation based on near-infrared absorption, and FIG. 15D is a result images of a cell live & dead staining experiment of the complex according to an embodiment of the present invention.

FIG. 16A is a result graph of an intracellular fluorescence analysis at pH 7.4 of the complex according to an embodiment of the present invention, and FIG. 16B is a result graph of an intracellular fluorescence analysis at pH 5.4.

FIG. 17 is an intracellular fluorescence analysis (left) and fluorescence intensity graph (right) of the complex over time, according to an embodiment of the present invention.

FIG. 18A is an intracellular fluorescence analysis graph over time in a serum solution of the complex according to an embodiment of the present invention, and FIG. 18B is a fluorescence image in a serum solution of the complex according to an embodiment of the present invention.

FIG. 19 is result images (left) of analyzing in vivo fluorescence imaging over time of a mouse injected with the complex according to an embodiment of the present invention and a fluorescence intensity graph (right).

FIG. 20 is images of in vivo MR T1 imaging analysis of the complex according to an embodiment of the present invention (a). For comparison, results for phosphate buffer saline (PBS) (b) and fluorescent polydopamine nanoparticles (SFPNPs) (c) are illustrated together.

FIG. 21 is result images and a graph of confirming changes in temperature caused by near-infrared illumination in the body of a mouse injected with the complex according to an embodiment of the present invention, using a thermal imaging camera.

FIG. 22 illustrates results of a photothermal effect (a), a change in body weight (b), a change in tumor size (c), an appearance after photothermal treatment (d), and Hematoxylin & Eosin (H&E) staining of a tumor after photothermal treatment in a mouse injected with the complex according to an embodiment of the present invention.

FIG. 23 is a result of an appearance of a tumor after photothermal treatment of a mouse injected with the complex and PBS according to an embodiment of the present invention.

FIG. 24 is a result of confirming an appearance of a tumor using H&E staining after photothermal treatment of a mouse injected with the complex and PBS according to an embodiment of the present invention.

### BEST MODE FOR DISCLOSURE

Hereinafter, the present disclosure will be described in detail with reference to the accompanying drawings.

One aspect of the present invention provides a complex including fluorescent nanoparticles and a manganese salt.

The fluorescent nanoparticles may include inorganic-based fluorescent nanoparticles, organic-based fluorescent nanoparticles, organic-inorganic composite fluorescent nanoparticles, or mixtures thereof. The inorganic-based fluorescent nanoparticles include, but are not limited to, semiconductor nanoparticles (also referred to as inorganic quantum dots), nanophosphors, metal nanoparticles such as gold, silver, palladium, and the like. The organic-based fluorescent nanoparticles may include, but are not limited to, organic fluorescent dyes, organic phosphors dispersed in a resin matrix, organic monomolecular phosphors, and the like. The organic-inorganic composite fluorescent nanoparticles may include, but are not limited to, inorganic nanoparticles embedded with organic fluorescent nanoparticles.

In an embodiment, the fluorescent nanoparticles may include fluorescent polydopamine nanoparticles (also referred to as “spherical fluorescent polydopamine nanoparticles (SFPNPs)”) formed using an eco-friendly material, dopamine, as a precursor. Accordingly, the complex may exhibit an effect that may be used as an eco-friendly and biostable contrast agent, a cancer diagnostic agent, and a cancer treatment agent.

In an embodiment, the fluorescent nanoparticle may be an organic fluorescent nanoparticle or organic-inorganic composite fluorescent nanoparticle including a catechol-derived functional group on surface thereof.

A size of the fluorescent nanoparticle is not limited thereto, but may have a diameter (D₅₀) of 10 to 300 nm. The size of the fluorescent nanoparticle may be, for example, 10 to 300 nm, 50 to 250 nm, or 100 to 200 nm, and when the size of the fluorescent nanoparticle is in the range described above, there may be a favorable effect by an EPR effect in delivering a medicine to a tumor cell for medication delivery and treatment to the cancer cell.

The manganese salt is a salt containing manganese (Mn) element, and is not particularly limited thereto, provided that it is capable of being conjugated to the surface of the fluorescent nanoparticle to quench the fluorescence of the fluorescent nanoparticle, as described below.

The manganese salt may include, but is not limited to, manganese carbonate (MnCO₃), manganese monoxide (MnO), manganese sulfate (MnSO₄), manganese chloride (MnCl₂), manganese nitrate (Mn(NO₃)₂, manganese acetate ((CH₃COO))₂Mn), or a mixture thereof.

In an embodiment, the manganese salt may include manganese carbonate (MnCO₃).

In an embodiment, the manganese salt may include a crystalline form.

The manganese salt may be conjugated to the surface of the fluorescent nanoparticle.

The term “conjugation” is used herein as a concept that includes both physical and chemical interactions, such as a covalent bond, an ionic bond, a hydrophilic-hydrophilic interaction, a hydrophobic-hydrophobic interaction, and bonding in complex compound. Accordingly, the “conjugation” of the manganese salt and the surface of the fluorescent nanoparticle may include physical and chemical interactions, such as a covalent bond, an ionic bond, a hydrophilic-hydrophilic interaction, a hydrophobic-hydrophobic interaction, and bonding in complex compound, between the manganese salt and the surface of the fluorescent nanoparticle.

Due to the conjugation of the manganese salt to the surface of the fluorescent nanoparticle, the fluorescent nanoparticle may be in a state of quenched fluorescence. In the complex, the present invention is not limited to a specific form of conjugation, provided that the fluorescence of the fluorescent nanoparticle may be quenched by the conjugation.

In an embodiment, the conjugation may include a chelating bond between a surface functional group of the fluorescent nanoparticle and the manganese salt, more specifically a manganese ion.

In an embodiment, the surface of the fluorescent nanoparticle may include a catechol-derived functional group, and the complex may include the manganese ion in the manganese salt being chelated by the catechol-derived functional group. Specifically, the conjugation may include a chelating bond of the manganese ion by an oxygen atom of an alcoholic group (—OH) of the 1,2-position of the catechol present on the surface of the fluorescent nanoparticle.

In an embodiment, the complex may have, for example, an average particle diameter (D₅₀) of less than 300 nm, and when the average particle diameter of the complex is 300 nm or more, there may be a problem in that efficient medication delivery and treatment effectiveness due to the EPF effect may be reduced, but the effect of the present invention is not limited thereto.

In a specific embodiment of the present invention, it was confirmed that the fluorescence of a fluorescent carbon nanoparticle is quenched by conjugation with a manganese ion (Mn²⁺).

In the complex, a content ratio of the fluorescent nanoparticle and the manganese salt may be such that, based on 1 mg/mL of the fluorescent nanoparticle in aqueous solution, the manganese ion (Mn²⁺) is included from 1 mM to 300 mM, but is not limited thereto. When the content ratio of the fluorescent nanoparticle and the manganese salt is in the range described above, it is possible to exhibit an effect of excellently improving an accuracy of bioimaging by the complex by increasing the degree of quenching based on maximum fluorescence intensity of the fluorescent nanoparticle.

In an embodiment, even if the concentration of manganese ion (Mn²⁺) is 300 mM or more based on 1 mg/mL of the fluorescent nanoparticle in the complex, there may be no difference in a fluorescence quenching rate, which may be an upper limit in terms of the efficiency of the complex.

In the complex, under a certain condition, the manganese salt may be ionized, thereby resulting in the recovery of fluorescence by the fluorescent nanoparticle.

The complex includes the fluorescent nanoparticle exhibiting fluorescence characteristics, but the fluorescence is quenched by conjugation of the manganese salt on the surface of the fluorescent nanoparticle, and the fluorescent nanoparticle recovers the fluorescence by ionization of the manganese salt under a certain condition, thereby having an effect of providing a bioimaging result targeting a detection target.

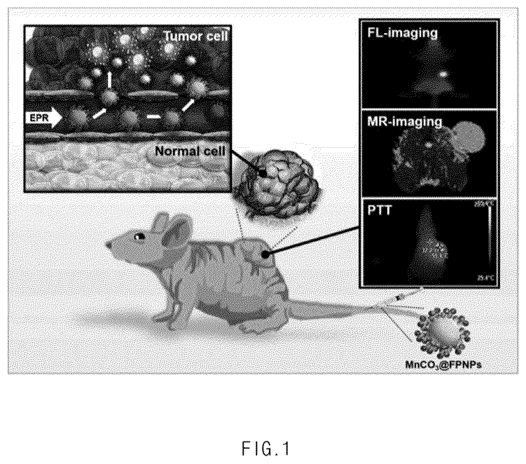

As illustrated in FIG. 1, the complex has an effect of enabling high-sensitivity diagnosis of a cancer cell by multiple bioimaging such as fluorescence imaging and MRI imaging that targets the cancer cell by the enhanced permeability and retention effect (EPR effect) of the macromolecule by the cancer cell. Simultaneously, the complex has a feature of absorbing light of near-infrared wavelength (approximately 808 nm) and releasing thermal energy. Therefore, there is an effect that the complex can be used as a multifunctional material of theragnosis that enables the treatment of cancer cell by a localized photothermal effect.

For example, the complex may recover the fluorescence under a condition in which the manganese salt is ionized to release the manganese ion from the complex.

In an embodiment, the ionization condition of the manganese salt may be an acidic condition. The acidic condition may refer to any condition of less than pH 7, and may refer specifically to a condition in which the cancer cell to be diagnosed is present, for example, a condition of a range of pH 6.5 or less, specifically a range of pH 6.37 or less, a range of pH 4 to pH 6, a range of pH 5 to pH 6, a range of pH 5 to pH 5.5, or pH 5.4. Accordingly, the complex may recover the fluorescence due to the ionization of the manganese salt in an environment where the cancer cell is present, thereby enabling bioimaging to target the cancer cell, but the mechanism of the present invention is not limited thereto.

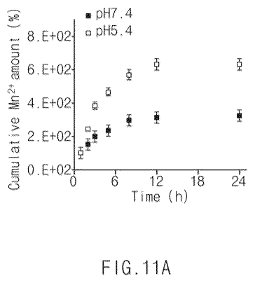

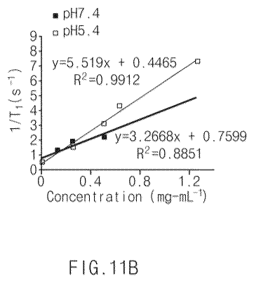

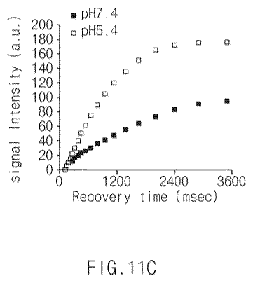

In a specific embodiment of the present invention, it was confirmed that the complex releases the manganese ion under acidic conditions, thereby causing a change in the magnetic properties of the complex and the recovery of fluorescence (FIGS. 11 to 13).

In addition, the complex may release thermal energy when illuminated with light. Specifically, the complex may release thermal energy when illuminated with light before, after, and simultaneously with the ionization of the manganese salt under a certain condition.

When the complex is illuminated with light, free electrons of the manganese salt in the Specifically, in the complex, the manganese complex may resonate to generate heat, salt may be ionized under a certain condition to release the manganese ion, and then, upon illumination with light, the free electrons of the released manganese ion resonate and dissipate heat to the surroundings, but the mechanism of the present invention is not limited thereto.

The light may be suitably selected by those skilled in the art depending on a specific kind of fluorescent nanoparticle and manganese salt, and may be, for example, light of near-infrared wavelength.

In an embodiment, the complex may absorb light in the near infrared at a wavelength of 780 to 850 nm, thereby generating heat by the resonance of free electrons in the manganese salt.

In an embodiment, the complex may include a manganese salt conjugated to the SFPNPs, specifically MnCO₃. In this case, the complex may absorb light of a wavelength of, for example, 808 nm to generate heat.

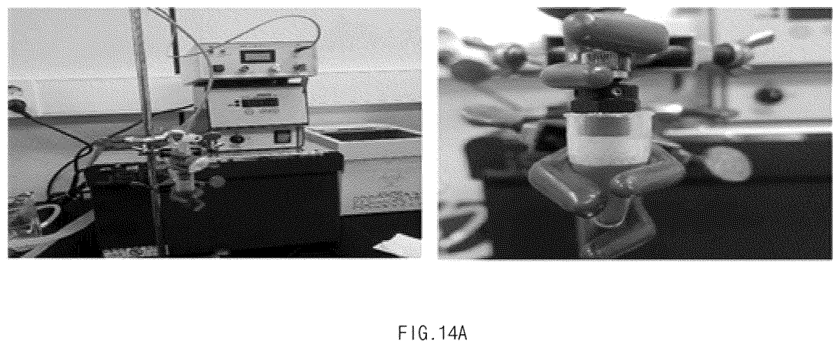

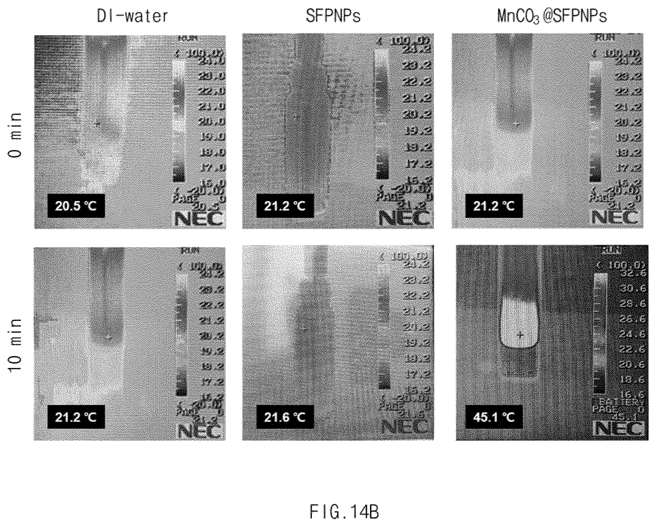

In a specific embodiment of the present invention, it was confirmed that when a solution including the complex is illuminated with light of near-infrared wavelength, the temperature of the solution increases over time (FIG. 14B).

In the present invention, the term complex is a concept that includes not only the complex itself, which includes the fluorescent nanoparticle and the manganese salt conjugated to the surface of the fluorescent nanoparticle, but also all the pharmaceutically acceptable salt, solvate, and hydrate forms thereof, without prejudice to the object of the present invention.

Due to the characteristics described above, the complex can be used as an active ingredient in a composition for bioimaging or in a composition for the diagnosis or treatment of cancer.

Accordingly, there is provided a composition for bioimaging including the complex in another aspect of the present invention.

In addition, there is provided a composition for cancer diagnosis including the complex in still another aspect of the present invention.

As described above, the complex includes the fluorescent nanoparticle, but is present in a state in which the fluorescence is quenched by the manganese salt conjugated to the surface of the fluorescent nanoparticle. Further, when the complex is exposed to a certain condition, the manganese salt is ionized from the complex, and the manganese ion is separated from the fluorescent nanoparticle, thereby causing the complex to recover the fluorescence. The complex has an effect that enables bioimaging by quenching of the fluorescence and specific recovery of the fluorescence. Further, there is an effect that bioimaging with MRI is possible due to the magnetic properties of the released manganese ion.

In this case, when the manganese salt is ionized under a condition in which cancer is present, there is an effect that the cancer can be diagnosed by bioimaging using the complex.

The bioimaging may be performed by a PET imaging method, an optical imaging method, an MRI imaging method, an optoacoustic method, a PET-MRI fusion imaging method, a NIRF-MRI fusion imaging method, a NIRF-PET fusion imaging method, a NIRF-CT fusion imaging method, and the like, and the complex may be used for bioimaging by various methods.

In an embodiment, the composition for bioimaging may be a composition for PET-MRI multi-imaging.

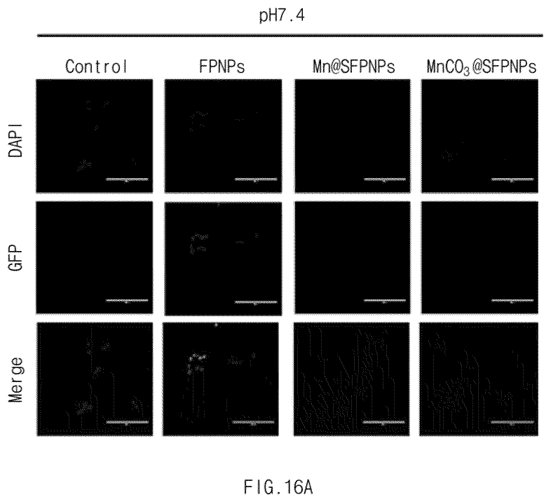

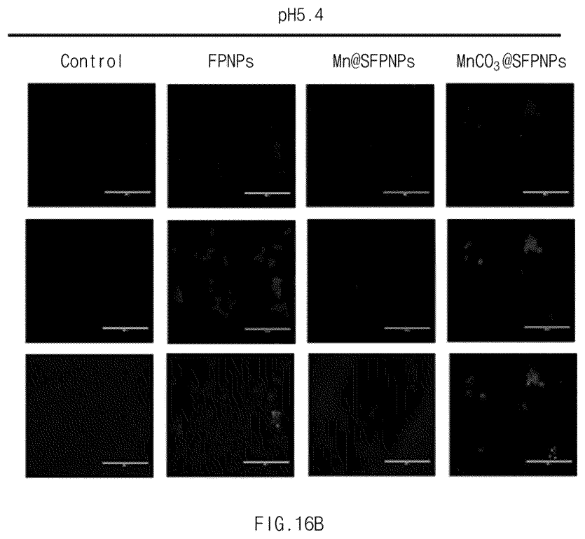

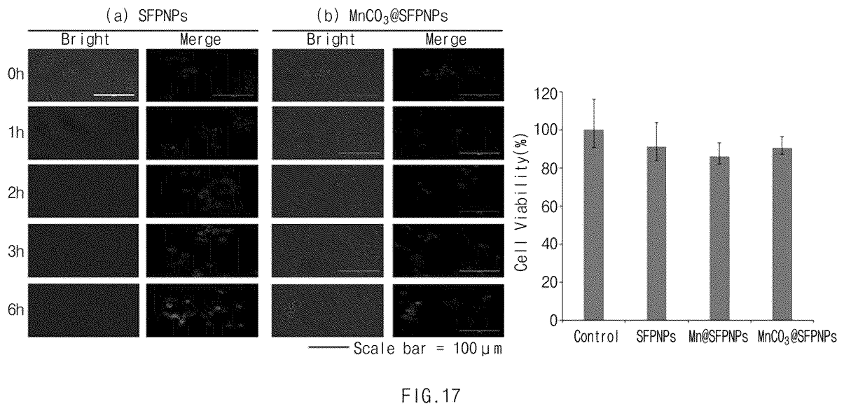

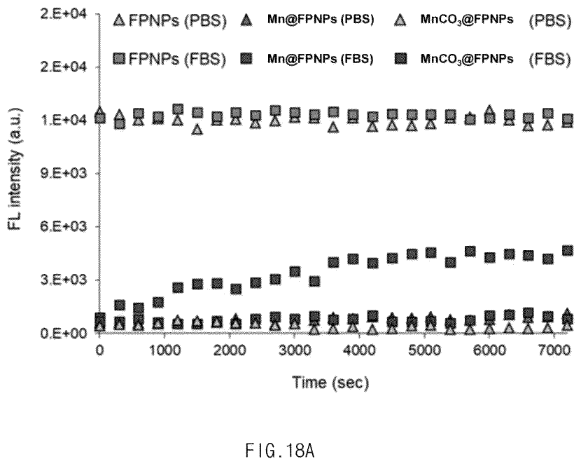

In a specific embodiment of the present invention, it was confirmed through an in vitro experiment that a cell treated with the complex maintained a quenched state of fluorescence under a neutral condition, while the quenched fluorescence was recovered under an acidic condition, thereby confirming that bioimaging using the complex is possible (FIGS. 16 and 17).

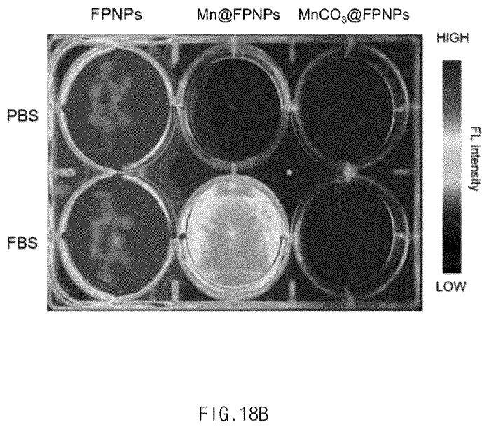

In a specific embodiment of the present invention, it was confirmed through an in vitro experiment that the complex maintained a quenched state of fluorescence in a serum solution, thereby confirming that bioimaging is possible in vivo using the complex (FIGS. 18A and 18B).

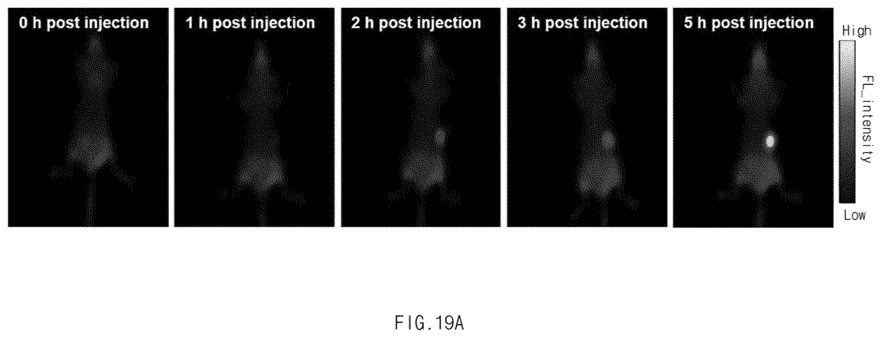

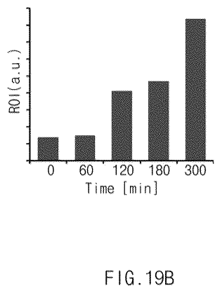

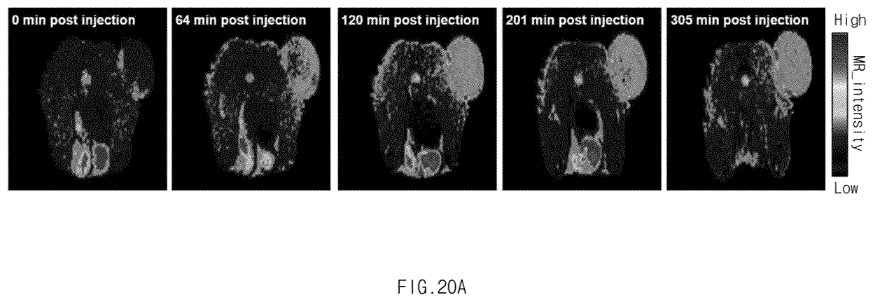

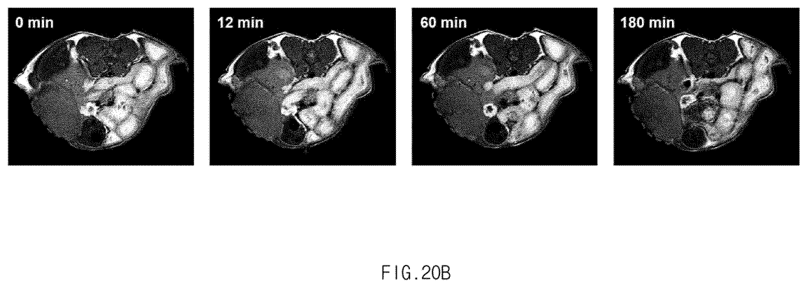

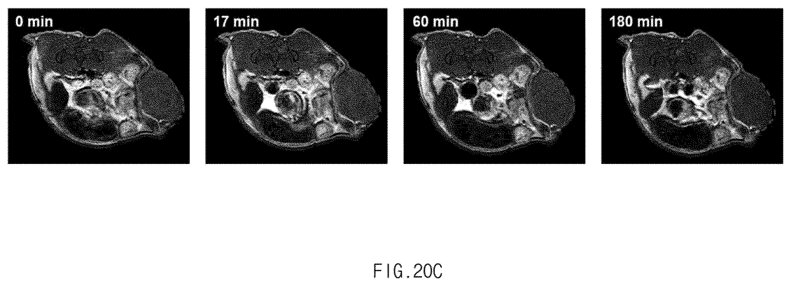

In another specific embodiment of the present invention, it was confirmed through an in vivo experiment that the fluorescence was specifically recovered in a certain site in the mouse injected with the complex (FIGS. 19 and 20).

There is provided a composition for prevention or treatment of cancer including the complex in still another aspect of the present invention.

As described above, the complex may release thermal energy when illuminated with light. Accordingly, in a photothermal treatment of a cancer cell, the use of the complex may further enhance photothermal heating by the illumination of light, specifically by the illumination of light of a specific wavelength, thereby exhibiting an effect of inhibiting the growth of the cancer cell or further causing and/or promoting the death of the cancer cell. The complex may exhibit a synergistic effect on the photothermal treatment by releasing thermal energy by the illumination of light.

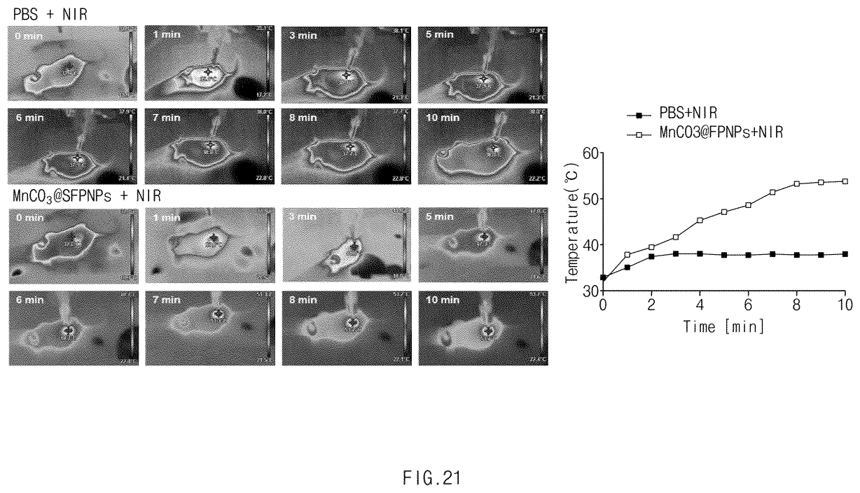

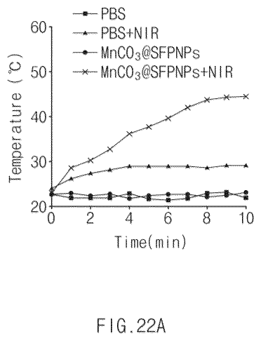

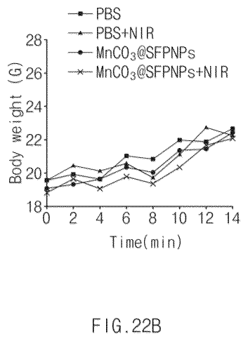

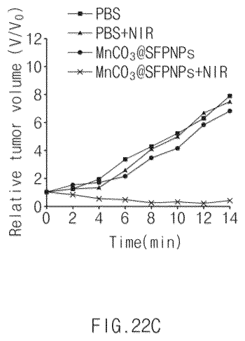

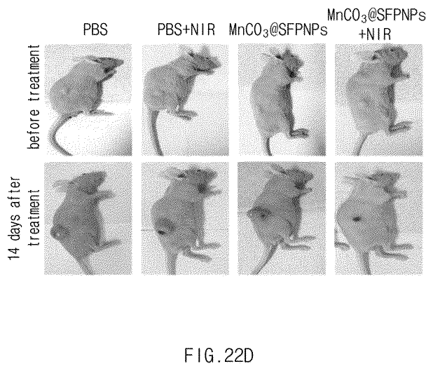

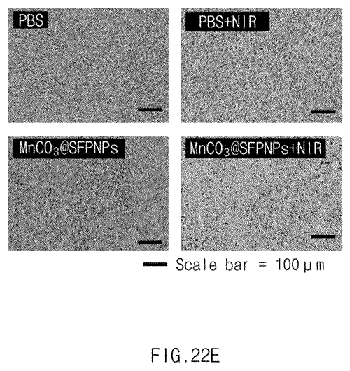

In a specific embodiment of the present invention, it was confirmed that when the complex was administered to a mouse and then illuminated with near-infrared light, there was a significant increase in the degree of temperature increase in a position-specific manner (FIG. 21). Further, it was confirmed that the size of the cancer cells of the mouse illuminated with near-infrared light was significantly reduced after the administration of the complex (FIG. 22).

A content of the complex in the composition may be, for example, 0.05 mg/mL to 5 mg/mL, specifically 0.1 mg/mL to 1 mg/mL, 0.3 mg/mL to 0.8 mg/mL, 0.3 mg/mL to 0.5 mg/mL, or 0.5 mg/mL, but is not limited thereto. When the content of the complex is in the range above, it is possible to exhibit an effect of significantly increasing the degree of increase in temperature when the complex is illuminated with light.

The composition for bioimaging and the compositions for the diagnosis, prevention, or treatment of cancer may further include a pharmaceutically acceptable carrier as necessary when formulated.

The composition for bioimaging and the compositions for the diagnosis, prevention, or treatment of cancer may be orally or parenterally administered in clinical administration and may be used in the form of a conventional pharmaceutical formulation. The compositions may further include a pharmaceutically acceptable carrier or additive and, when formulated, may be prepared using a commonly used diluent or excipient such as a filler, extender, binder, humectant, disintegrant, surfactant, or the like.

There is provided a method of diagnosing cancer including a step of injecting the complex in still another aspect of the present invention.

In addition, there is provided a method of preventing or treating cancer including the step of injecting the complex in yet another aspect of the present invention.

The method of preventing or treating cancer further include a step of illuminating with light.

In the present invention, the cancer to be diagnosed, prevented or treated may include, for example, at least one selected from the group consisting of breast cancer, liver cancer, lung cancer, colorectal cancer, gastric cancer, colon cancer, bone cancer, pancreatic cancer, head or neck cancer, uterine cancer, ovarian cancer, rectal cancer, esophageal cancer, small intestinal cancer, anorectal cancer, colon cancer, fallopian tube carcinoma, endometrial carcinoma, cervical carcinoma, vaginal carcinoma, vulvar carcinoma, Hodgkin's disease, prostate cancer, bladder cancer, kidney cancer, vesicoureteral cancer, renal cell carcinoma, renal pelvic carcinoma, and central nervous system tumors, but is not limited thereto.

### Mode for Disclosure

Hereinafter, the present invention will be described in detail with reference to the drawings and examples. Terms or words used in the specification and the claims should not be interpreted as being limited to a general or dictionary meaning and should be interpreted as a meaning and a concept which conform to the technical spirit of the present invention based on a principle that an inventor can appropriately define a concept of a term in order to describe his/her own invention by the best method.

FIG. 1 illustrates a schematic view of a cancer cell treatment mechanism by bioimaging and photothermal therapy of a complex of according to an embodiment of the present invention.

It was confirmed that there is an effect of being capable of diagnosing cancer using the complex of the present invention and an effect of treating a cancer cell by photothermal therapy, as described in the following examples.

### [Example 1] Preparation of Fluorescent Nanoparticle-Metal Manganese Salt Complex

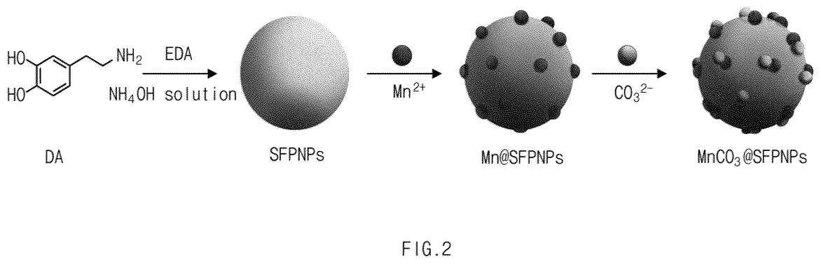

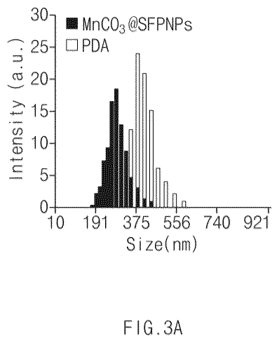

According to the schematic view illustrated in FIG. 2, the complex according to an embodiment of the present invention was prepared as follows.

### [1-1] Synthesis of Dopamine-Derived Fluorescence Nanoparticles (SFPNPs)

Spherical fluorescent polydopamine nanoparticles (SFPNPs) exhibiting fluorescence were prepared by adding 200 mg of dopamine and 1 mL of ethylenediamine (purity of 99.5% or more (GC)) to 60 mL of ammonia water solution formulated with 5 mL of 28% ammonia water, 80 mL of 98% ethanol and 180 mL of distilled water and then allowing the reaction to proceed for 12 hours at room temperature with stirring at a speed of 300 RPM.

### [1-2] Formation of Mn@SFPNPs Intermediate Complex

The SFPNPs prepared in Example [1-1] above were purified by centrifugation at a speed of 10,000 RPM and dialysis for two days. Manganese-fluorescent polydopamine nanoparticles (Mn@SFPNPs) were prepared by reacting 10 mL of the purified 1 mg/mL concentration of SFPNPs with 100 mM concentration of manganese ions (Mn²⁺) for 12 hours followed by the purification with an osmotic process.

### [1-3] Formation of MnCO₃@ FPNPs Complex

The complex (MnCO₃@SFPNPs) of manganese carbonate-fluorescent polydopamine nanoparticles was prepared by reacting the manganese-fluorescent polydopamine nanoparticles (Mn-SFPNPs) prepared in Example [1-2] above with 100 mM of disodium carbonate (Na₂CO₃) aqueous solution, followed by osmotic purification and freeze-drying.

### [Example 2] Structural Analysis of Fluorescent Nanoparticle-Metal Manganese Salt Complex

### [2-1] Structural Analysis 1 of Complex

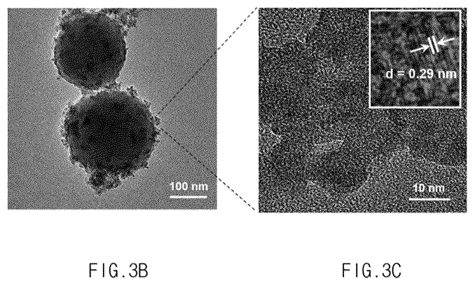

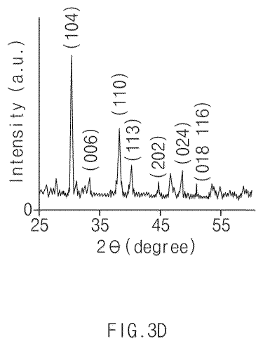

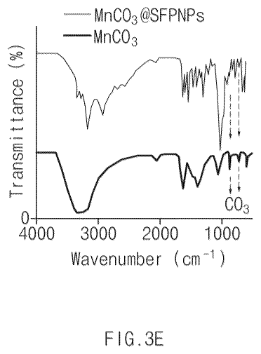

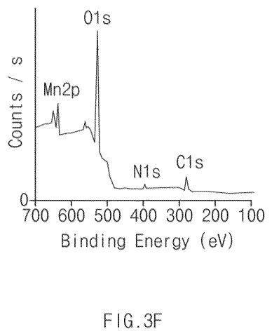

For polydopamine (PDA) prepared from 200 mg of dopamine and an aqueous solution of ammonia, and for each of the materials prepared in Example [1-1], Example [1-2], and Example [1-3] above, dynamic light scattering (DLS, Beckman Coulter), transmission electron microscopy (TEM, G2 F30X-TWIN, 300 kV), X-ray diffraction (XRD, Rigaku-Ultima IV), FT-IR (Bruker Corp., Billerica, MA, USA), and X-ray photoelectron spectrometer (XPS, Thermo Fisher Waltham, MA, USA) were measured, respectively, and the results are illustrated in FIG. 3.

(a) From the DLS analysis result, it was confirmed that in the complex (MnCO₃@SFPNPs) of Example [1-3], the fluorescent nanoparticles (SFPNPs) are structurally degraded by EDA compared to PDA. It was confirmed by the crystal structure that (b) the MnCO₃@SFPNPs complex prepared from dopamine has a spherical shape from the TEM result, and (c) the manganese carbonate is immobilized on the surface of SFPNPs using the high magnification TEM result. With further crystal structure analysis, (d) it was confirmed that the complex (MnCO₃@SFPNPs) has the same crystal structure as manganese carbonate from the XRD analysis result, and (e) it was confirmed that the bending vibration peak of carbon dioxide, which is not seen in the fluorescent nanoparticles (SFPNPs) itself prepared from dopamine, was generated in the MnCO₃@SFPNPs complex by the FT-IR result, thereby confirming that MnCO₃ was bound to the surface of the fluorescent nanoparticles. In addition, (f) it was confirmed whether the manganese ions in the complex (MnCO₃@SFPNPs) were present by the XPS survey analysis.

### [2-2] Structural Analysis 2 of Complex

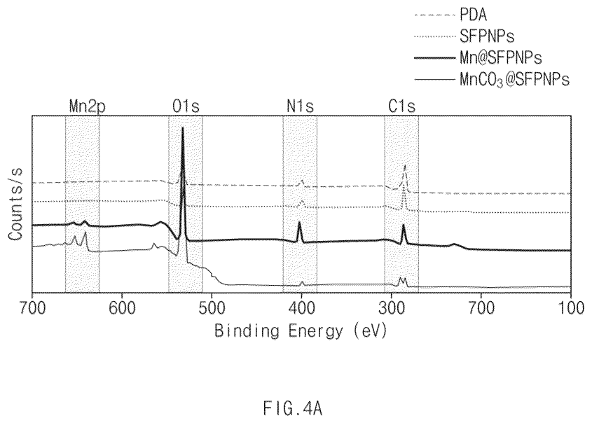

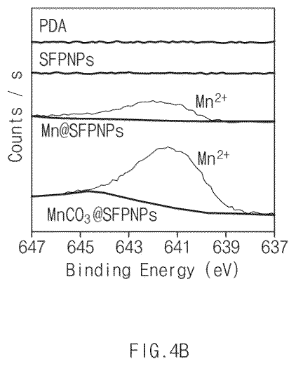

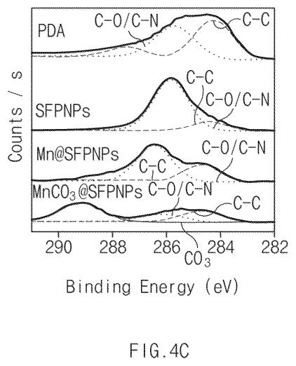

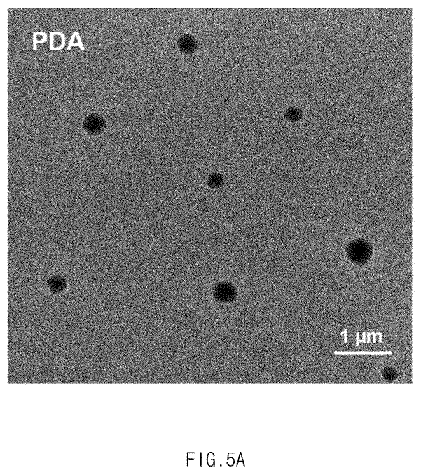

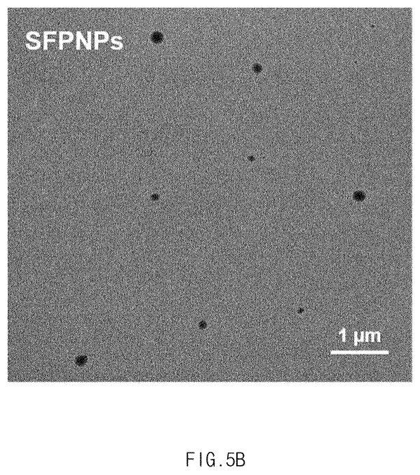

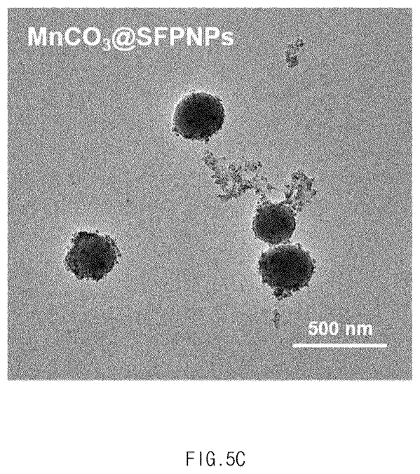

Using the X-ray photoelectron spectroscopy (XPS, Thermo Fisher Waltham, MA, USA), the interatomic binding energy of each of polydopamine (PDA) nanoparticles, SFPNPs, Mn@SFPNPs intermediate complex and MnCO₃@SFPNPs complex was analyzed by (a) survey scan and (b and c) narrow scan, and the results are illustrated in FIG. 4. Here, the PDA nanoparticles means polydopamine nanoparticles prepared according to the conventional polymerization method.

In analyzing the results in FIG. 4, (a) the binding energy with the manganese element that was not observed for the SFPNPs, which are the polydopamine (PDA) nanoparticles and dopamine-derived fluorescent nanoparticles, in the Mn@SFPNPs intermediate complex and the MnCO₃@SFPNPs complex of the survey scan, was confirmed. In addition, (c) the binding energy with carbonic acid (CO₃²⁻) in the 289 eV region is confirmed for the MnCO₃@SFPNPs complex in the narrow scan.

From the results described above, it was confirmed that the MnCO₃@SFPNPs complex according to the present invention includes a structure in which MnCO₃ is formed on the SFPNPs.

### [2-3] Structural Analysis 3 of Complex

The complex (MnCO₃@ FPNPs) prepared in Example [1-3] above was observed by transmission scanning microscopy (TEM) and the structure thereof was analyzed. From the observation of the structure of each of the SFPNPs, Mn@SFPNPs intermediate complex, and MnCO₃@SFPNPs complex, it was confirmed that the complex of Example [1-3] above has a crystalline structure that was not seen in the conventional FPNPs and Mn@SFPNPs intermediate complex as the manganese salt of MnCO₃ was conjugated to the surface of the SFPNPs.

### [2-4] Structural Analysis 4 of Complex

Each element constituting nanoparticles in the MnCO₃@SFPNPs complex was mapped using TEM analysis equipment, and the results are illustrated in FIG. 6.

According to FIG. 6, the manganese ions (Mn, in yellow) were detected in the MnCO₃@SFPNPs, and it was confirmed that the manganese ions were present.

### [Example 3] Analysis of Characteristics of Fluorescent Nanoparticle-Metal Manganese Salt Complex—In Vitro

### [3-1] Analysis 1 of Optical Characteristics of Complex

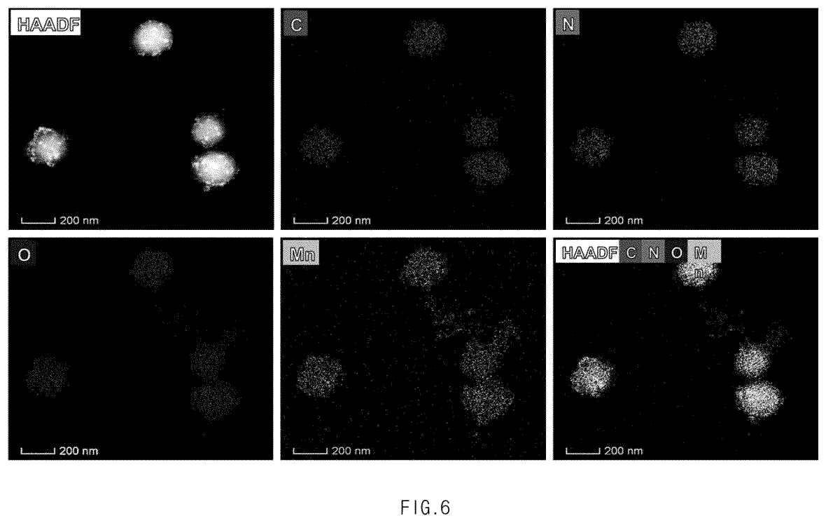

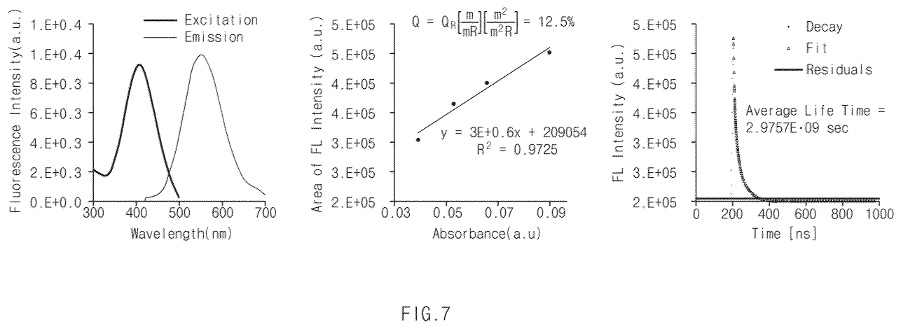

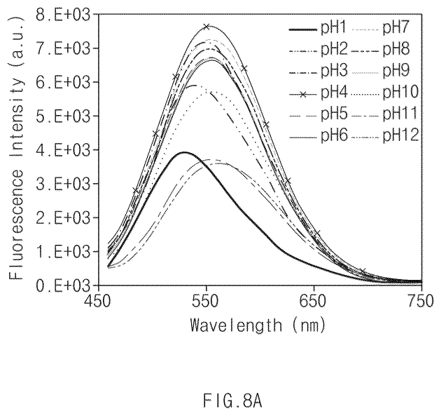

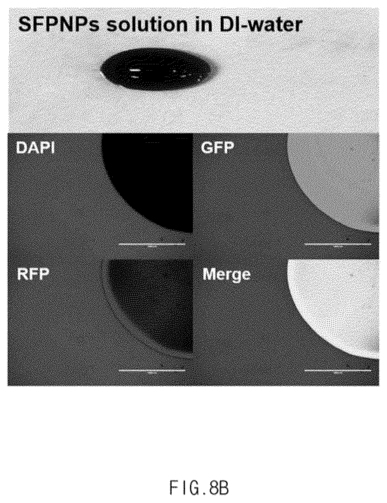

The fluorescence spectra, quantum efficiency, and fluorescence lifetime of Example [1-1] (SFPNPs) above were measured using a fluorescence spectrometer (Scinco FluoroMate FS-2, Seoul, South Kore), and the results are illustrated in FIG. 7. In addition, the excitation wavelength-dependent broad fluorescence emission wavelength and fluorescence stability from pH 2 to pH 10 of Examples [1-1] were confirmed, and the results are illustrated in FIG. 8.

Specifically, according to FIG. 7, it was confirmed that the SFPNPs exhibit fluorescence by being excited at a wavelength of 400 nm or more and emitting an excitation wavelength at 560 nm. Further, it was confirmed that the fluorescence yield and fluorescence lifetime were 12.5% and 2.9 ns, respectively.

In addition, according to FIG. 8, it was confirmed that the SFPNPs have the characteristics of absorbing and emitting light in a wide range of wavelengths, such as in blue (380 nm or more), green (420 nm or more), and red (550 nm or more), and have a high stability to light under all pH conditions.

Therefore, it was confirmed that imaging in acidic and low-oxygen environments of a cancer cell can be achieved without a change in the fluorescence wavelength using the fluorescent nanoparticles of Example [1-1] above.

### [3-2] Analysis 2 of Optical Characteristics of Complex—Confirmation of Quenching Effect

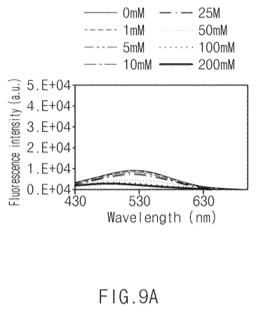

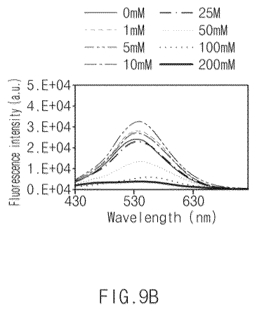

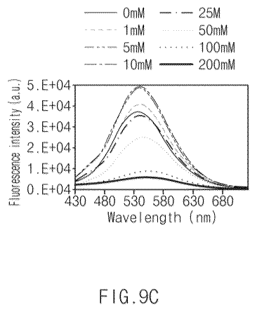

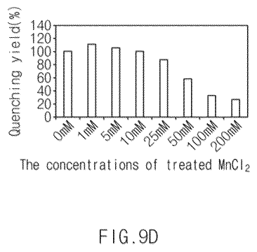

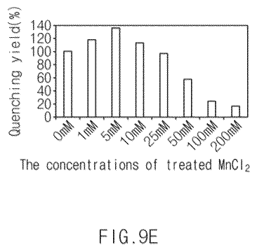

The degree of quenching of fluorescence (quenching yield, %) by the manganese ions of the SFPNPs and the degree of fluorescence emission using fluorescence spectroscopy were measured, by preparing the aqueous solutions of SFPNPs with concentrations of (a) 0.1 mg-mL⁻¹, (b) 0.5 mg-mL⁻¹, and (c) 1 mg-mL⁻¹, respectively, of the SFPNPs of Example [1-1] above, in which 0 mM, 1 mM, 5 mM, 10 mM, 25 mM, 50 mM, 100 mM, or 200 mM of manganese ions (Mn²⁺) were added, respectively, and the results are illustrated in FIG. 9.

With reference to FIG. 9, it was confirmed that since the SFPNPs are nanoparticles with a catechol functional group on the surface thereof, the metal ions may be chelated due to the oxidation of the catechol group, and the SFPNPs coupled with manganese metal ions (Mn-SFPNPs) exhibit dynamic and static quenching under the influence of the metal ions.

Meanwhile, it was confirmed that the degree of quenching depends on the concentration of fluorescent nanoparticles and manganese. In particular, it was confirmed that the fluorescence was quenched to 10 to 25% of the original fluorescence at a maximum upon addition of 200 mM of manganese ions when the concentration of SFPNPs was (a) 0.1 mg-mL⁻¹, (b) 0.5 mg-mL⁻¹, and (c) 1 mg-mL⁻¹, respectively.

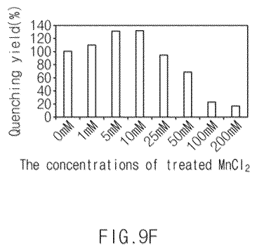

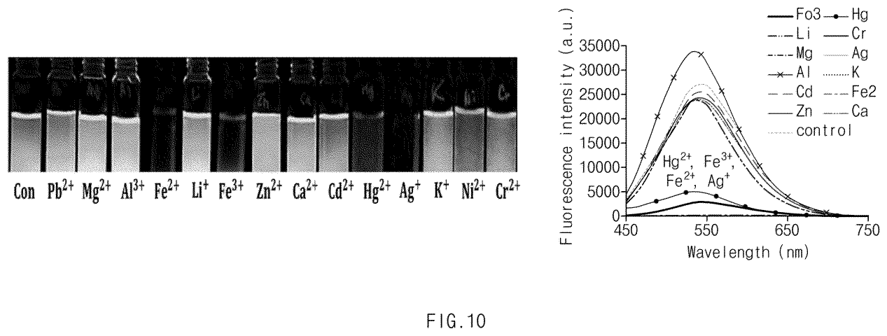

FIG. 10 illustrates the results of confirming that the SFPNPs may exhibit the quenching effect by other metal ions (iron (Fe²⁺ and Fe³⁺), mercury (Hg²⁺), silver (Ag⁺), etc.) in addition to the manganese ion. Therefore, it was confirmed that the complex (MnCO₃@SFPNPs) of Example [1-3] above may exhibit the quenching effect by chelation of the SFPNPs with the manganese ions in MnCO₃.

### [3-3] Analysis of Changes in Properties of the Complex with pH

Inductively coupled plasma optical emission spectroscopy (ICP-OES), changes in longitudinal magnetic field, and longitudinal magnetic field effect over time of the complex of Examples 1-3 above in an acidic condition of pH 5.4 and a neutral condition of pH 7.4, respectively, were measured with time and concentration, and the results are illustrated in FIG. 11.

From the ICP-OES graph in FIG. 11A, it is confirmed that in the MnCO₃@SFPNPs complex, the amount of manganese ions released increases over time in the acidic condition compared to the neutral condition. From the longitudinal magnetic field graph in FIG. 11B, it was confirmed that the MnCO₃@SFPNPs complex has an increasing longitudinal magnetic field effect with higher concentration thereof. From the recovery graph of the longitudinal magnetic field in FIG. 11C, it was confirmed that the longitudinal magnetic field effect of the MnCO₃@SFPNPs complex increases as the time elapses and becomes saturated at 2400 msec.

Therefore, it was confirmed that the MnCO₃@SFPNPs complex is ionized under the acidic condition and has a capability to release the manganese ions. In addition, it was confirmed that the released manganese ions have magnetic properties and that magnetic resonance (MR) T1 imaging can be implemented in vivo by longitudinal magnetization relaxation.

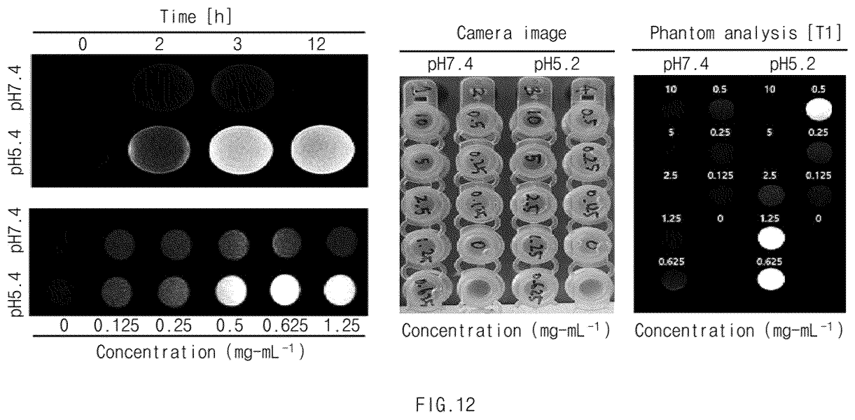

Accordingly, the MR phantom T1 imaging of the MnCO₃@SFPNPs complex was measured in an acidic condition of pH 5.4 and a neutral condition of pH 7.4, respectively, and the results are illustrated in FIG. 12. According to FIG. 12, it was confirmed that the MR phantom T1 imaging of the MnCO₃@SFPNPs complex is observed to be brighter as the reaction time of the MnCO₃@SFPNPs complex is longer in the solvent under the acidic condition (pH 5.4) from the upper image of FIG. 12, and the concentration of the MnCO₃@SFPNPs complex is higher from the lower image of FIG. 12.

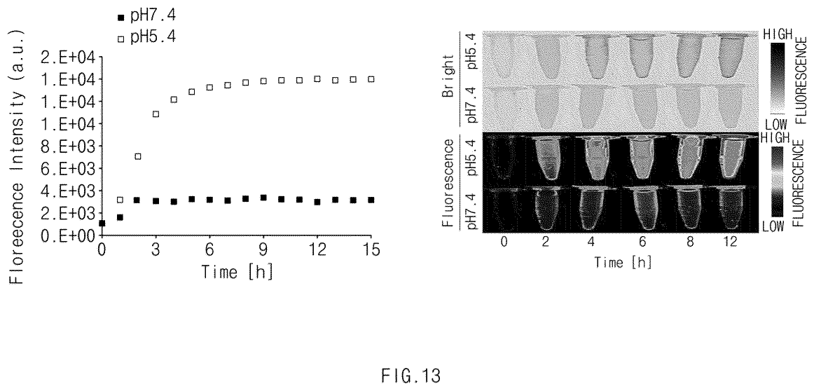

In addition, the recovery characteristic of the fluorescence with time were observed for the MnCO₃@SFPNPs complex in the acidic condition of pH 5.4 and the neutral condition of pH 7.4, respectively, and the results are illustrated in FIG. 13. According to FIG. 13, it was confirmed that the fluorescence characteristics of the fluorescent nanoparticles of the MnCO₃@SFPNPs complex were recovered as the manganese ions were ionized in the acidic condition, while the fluorescence characteristics were still quenched in the neutral condition.

Therefore, it was confirmed that the MnCO₃@SFPNPs complex can recover the fluorescence of fluorescent nanoparticles by the ionization of manganese ions under the acidic condition.

### [3-4] Analysis of Heat Dissipation Characteristic of Complex

An experiment was conducted to confirm the heat dissipation characteristic by near-infrared absorption of the complex of Example [1-3] above and the corresponding cell killing effect.

First, the complex prepared in Example [1-3] above was illuminated with near-infrared light at a wavelength of 808 nm at room temperature using a light source device coupled with an optical fiber, and the heat dissipated therefrom was measured with a thermal imaging camera (GTC 400 C, Bosch), and the result is illustrated in FIG. 14B.

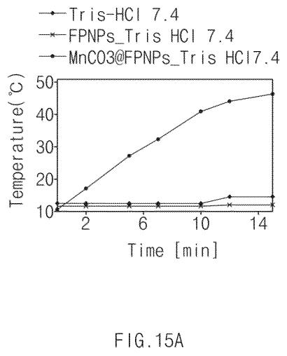

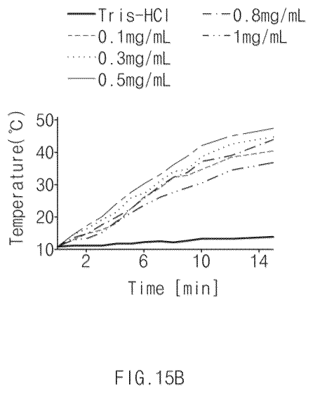

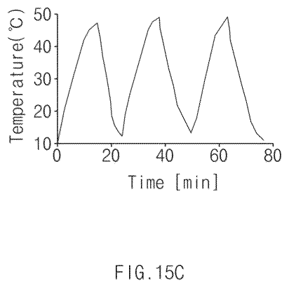

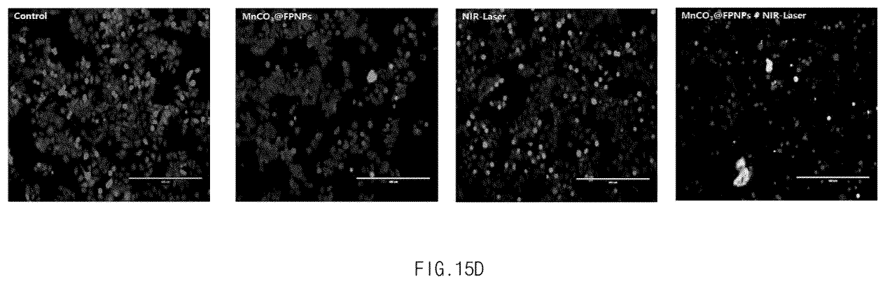

The effect of the heat dissipation characteristic was confirmed using Tris-HCl PH 7.4 solution as a control and SFPNPs in the Tris-HCl pH 7.4 solution (FIG. 15A), and also the change in temperature with the concentration of the complex of Example [1-3] (FIG. 15B) and the thermal stability of the complex according to repeated experiment (FIG. 15C) were confirmed, and the results are illustrated in FIG. 15.

According to FIG. 14B and FIG. 15A, it was confirmed that only the complex of Example [1-3] can specifically dissipate heat when illuminated by near-infrared light, and according to FIG. 15B, it was confirmed that the heat dissipation effect is the most excellent, particularly at a concentration of 0.5 mg/mL. In addition, according to FIG. 15C, it was confirmed that the complex has excellent thermal stability even when the experiment is repeatedly performed according to temperature conditions.

Next, MCF-7 cell was used to perform live & dead cell staining experiments before and after near-infrared illumination at a wavelength of 808 nm, respectively, for culture medium (control), and culture medium (MnCO₃@SFPNPs) in which the MnCO₃@SFPNPs complex solution was diluted to a concentration of 0.5 mg/mL, and the results are illustrated in FIG. 15D. In this case, the normal cell appears in green and the abnormal cell in red.

According to FIG. 15D, the normal cell was observed in both the culture medium (control) and the culture medium containing the MnCO₃@SFPNPs complex before the near-infrared illumination, while after the near-infrared illumination, only the normal cell was observed in the culture medium (NIR Laser), but the abnormal cell was observed in the culture medium containing the MnCO₃@SFPNPs complex, thereby confirming that the MnCO₃@SFPNPs complex can induce the cell death by the heat of the cell by the light of the near-infrared wavelength.

### [3-5] Intracellular Fluorescence and Toxicity Assessment 1 of Complex

The MnCO₃@SFPNPs complex of Example [1-3] above was dissolved in a solution under acidic or neutral conditions at a concentration of 0.5 mg/mL for 24 hours. The MCF-7 cell cultured in the culture medium (DMEM, FBS 10%, P/S 1%) for 24 hours was treated with each solution in which the complex was dissolved for one hour, DAPI staining was performed and then the cell was washed with PBS, and the fluorescence images of the cell were observed using a fluorescence microscope (EVOS, Thermo Fisher). The same experiments were performed for the SFPNPs and Mn@SFPNPs intermediate complex, and the observation results are illustrated in FIG. 16.

According to FIG. 16A, it was confirmed that SFPNPs in the unquenched state in the neutral solution (pH 7.4, 1 mM phosphate buffer) exhibited green fluorescence directly in the cytoplasm within two hours after the treatment of the cell, while the MnCO₃@SFPNPs complex did not exhibit fluorescence in the cell like Mn@SFPNPs because the manganese ions were not ionized but conjugated to the fluorescent nanoparticles and quenched the fluorescence.

In contrast, according to FIG. 16B, in the acidic solution (pH 5.4, 1 mM acetate buffer), in the MnCO₃@SFPNPs complex, the manganese ions were decomposed from the fluorescent nanoparticles in the quenched state and absorbed by the cell, and the fluorescence caused by the fluorescent nanoparticles was immediately visible in the cytoplasm.

### [3-6] Intracellular Fluorescence and Toxicity Assessment 2 of Complex

The MCF-7 cell cultured in the culture medium (DMEM, FBS 10%, P/S 1%) for 24 hours was treated with the complex (MnCO₃@SFPNPs) and SFPNPs of Example [1-3]above at a concentration of 0.5 mg/mL respectively, then stained with DAPI and washed with PBS, and the change of intracellular fluorescence characteristics with time was observed by fluorescence microscopy, and the results are illustrated on the left side of FIG. 17.

With reference to the left drawing of FIG. 17, it was confirmed that (a) for the SFPNPs, the fluorescence characteristics caused by the SFPNPs absorbed into the cytoplasm are observed in the GFP region after one hour elapsed after the treatment, but (b) for the MnCO₃@SFPNPs complex, the intracellular fluorescence is observed after three hours elapsed after the treatment of the complex due to the ionization of manganese ions after the formation of the acidic condition by the endosome.

Next, the MCF-7 cell cultured in the culture medium (DMEM, FBS 10%, P/S 1%) for 24 hours was treated with the SFPNPs, Mn@SFPNPs intermediate complex, and MnCO₃@SFPNPs complex, respectively, at a concentration of 0.5 mg/mL, and the cytotoxicity was then assessed by measuring the absorbance at 450 nm using the CCK-8 kit. The results of confirming the cytotoxicity are illustrated on the right side of FIG. 17.

With reference to the right drawing of FIG. 17, it was confirmed that the SFPNPs, Mn@SFPNPs, and MnCO₃@SFPNPs all have a stable effect on the cytotoxicity. In particular, the SFPNPs, which are dopamine-derived eco-friendly materials, exhibit stable cytotoxicity, and the complex of Example [1-3] conjugated with MnCO₃ manganese salt also exhibits the same or similar level of cytotoxicity. Therefore, it was confirmed that according to the present invention, it is possible to provide materials useful for bioimaging of cells with excellent stability in the body and for diagnosis or treatment of cancer cells.

### [3-7] Serum Stability Assessment of Complex

To confirm the serum stability of the MnCO₃@SFPNPs complex, the MnCO₃@SFPNPs complex of Example [1-3] above was dissolved in the PBS buffer or fetal bovine serum (FBS) solution at a concentration of 0.5 mg/mL for 24 hours, and the fluorescence images of the cell were observed using the fluorescence microscope (EVOS, Thermo Fisher). The same experiments were performed for the SFPNPs and Mn@SFPNPs intermediate complex, and the observation results are illustrated in FIG. 18A and FIG. 18B for the time-dependent fluorescence spectra and fluorescence images, respectively.

According to FIG. 18A and FIG. 18B, it was confirmed that the SFPNPs in the unquenched state exhibit fluorescence immediately after the treatment, the Mn@SFPNPs intermediate complex exhibits increased fluorescence due to decreased serum stability over time in the FBS solution, while the MnCO₃@SFPNPs complex does not exhibit any fluorescence. These results suggest that the MnCO₃@SFPNPs complex, as the surface thereof is mineralized, exhibits higher serum stability compared to the Mn(q SFPNPs complex, and therefore, when applied in vivo, only generates fluorescence in the presence of cancer cells, enabling reliable bioimaging of cells.

### [Example 4] Analysis of Characteristics of Fluorescent Nanoparticle-Metal Manganese Salt Complex—In Vivo

### [4-1] Intracellular Multiplexed Imaging Assessment 1 of Complex

The MCF-7 was xenografted into a 5-week-old balb/c nude mouse (19 to 21 g) through subcutaneous (SC) injection. In the mouse xenografted with a tumor of size of 80 mm³ as described above, the sterilized MnCO₃@SFPNPs complex was injected intravenously (IV) into the tail of the mouse at a concentration of 0.5 mg/mL, and the biological fluorescence images over time were observed using an IVIS (in vivo imaging system) equipment, and the results are illustrated in FIG. 19.

According to the results in FIG. 19, it was confirmed that the mouse injected with the MnCO₃@SFPNPs complex exhibited no initial fluorescence imaging in the normal cell, but in the acidic environment where the cancer cell was present, fluorescence was observed from two hours after the injection.

Therefore, it was confirmed that according to the present invention, it is possible to specifically detect (diagnose) cancer cells through biological fluorescence imaging.

### [4-2] Intracellular Multiplexed Imaging Assessment 2 of Complex

The 5-week-old balb/c nude mouse with 4T1 cancer cell grown to a size of 80 mm³ was injected intravenously with phosphate buffer saline (PBS) of pH7.4, SFPNPs of a concentration of 0.5 mg/mL, and MnCO₃@SFPNPs complex of a concentration of 0.5 mg/mL, each in a volume of 0.1 mL into the tail of the mouse. After the injection, T1 magnetic resonance imaging (MRI) was analyzed over time and the results are illustrated in FIG. 20.

According to FIG. 20A, it was confirmed that in the mouse injected with the MnCO₃@SFPNPs complex from two hours after the injection, MR T1 imaging was observed through longitudinal magnetization relaxation in the cancer cell as a result of the release of manganese ions from the MnCO₃@SFPNPs complex due to the acidic environment within the cancer cell, while according to FIG. 20B and FIG. 20C, in the mouse injected with the PBS and SFPNPs, the manganese ions are not present in the cancer cell, so no magnetic T1 imaging due to the magnetization was observed.

Therefore, it was confirmed that according to the present invention, it is possible to specifically detect cancer cells through MRI analysis.

### [4-3] Thermal Characteristics In Vivo Assessment of Complex

The complex (MnCO₃@SFPNPs) of Example [1-3] above was injected intratumorally (IT) into a 6-week-old balb/c nude mouse with 4T1 cancer cell grown to a size of 80 mm³ at a concentration of 0.5 mg/mL, and the changes in body temperature by the illumination of near-infrared light were confirmed using a thermal imaging camera, and the results are illustrated in FIG. 21.

According to FIG. 21, it was confirmed that the body temperature of the mouse increased by the illumination of near-infrared light, in which case the degree of increase in temperature was significantly higher in the mouse illuminated with near-infrared light on the complex (MnCO₃@SFPNPs) of Example [1-3] compared to the mouse illuminated with near-infrared light on the PBS.

Therefore, it was confirmed that for the photothermal therapy of cancer cells using near-infrared illumination, the use of the complex of the present invention can significantly increase the degree of increase in body temperature, thereby exhibiting an excellent synergistic effect for the photothermal therapy.

### [4-4] Assessment 1 of In Vivo Cancer Cell Inhibition Effect of Complex

The MnCO₃@SFPNPs complex of Example [1-3] above was injected intratumorally (IT) into the 6-week-old balb/c nude mouse with 4T1 cancer cell grown to a size of 80 mm³ at a concentration of 0.5 mg/mL, and then illuminated with near-infrared light for 14 days to confirm the inhibition effect of the cancer cell, and the results are illustrated in FIG. 22.

First, compared to the PBS with the mouse illuminated with near-infrared light on the PBS, (a) changes in temperature, (b) changes in body weight, and (c) changes in the size of cancer cell in the MnCO₃@SFPNPs complex and the mouse illuminated with near-infrared light on the MnCO₃@SFPNPs complex were observed. As a result, as illustrated in FIG. 22A, it was confirmed that only the mouse illuminated with near-infrared light on the MnCO₃@SFPNPs complex exhibited an increase in temperature due to the photothermal effect, and as illustrated in FIG. 22C, a decrease in tumor size was observed due to the occurrence of tumor cell death by the photothermal effect, and the weight of the mouse did not change significantly, as illustrated in FIG. 22B. In addition, as illustrated in FIG. 22D, it was confirmed that the tumor size significantly decreased in the mouse illuminated with near-infrared light on the MnCO₃@SFPNPs complex after the photothermal treatment compared to the mice with MnCO₃@SFPNPs, PBS and illuminated with near-infrared light on the PBS, and it was confirmed of the cancer cell death through pathological examination with Hematoxylin & Eosin (H&E) staining. The results as described above suggest that the complex of the present invention not only has an effect of inhibiting the growth of cancer cells by generating photothermal heat due to the effect of absorbing light and dissipating heat, but also has an effect of inducing the cancer cells death.

### [4-5] Assessment 2 of In Vivo Cancer Cell Inhibition Effect of Complex

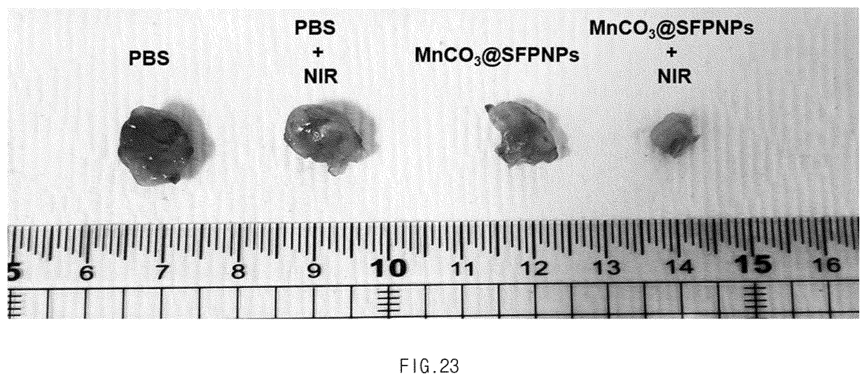

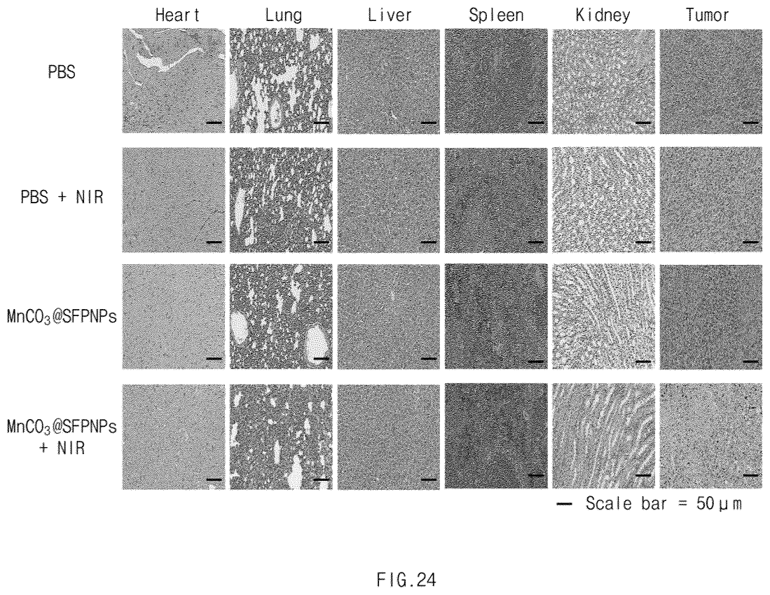

The MnCO₃@SFPNPs complex of Example [1-3] above was injected intratumorally (IT) into the 6-week-old balb/c nude mouse with 4T1 cancer cell grown to a size of 80 mm³ at a concentration of 0.5 mg/mL, and then irradiated with near-infrared light for 14 days to confirm the inhibition effect of the cancer cell, and the results are illustrated in FIGS. 23 and 24.

According to FIG. 23, it was confirmed that when the size of the tumor extracted from the mouse illuminated with near-infrared light after the injection of the MnCO₃(a SFPNPs complex of Example [1-3] was compared with the size of the tumor extracted from the mice with MnCO₃@SFPNPs, PBS, and illuminated with near-infrared light on the PBS, the size of the tumor extracted from the mouse illuminated with near-infrared light after the injection of the MnCO₃@SFPNPs complex significantly decreased. That is, the results as described above suggest that the complex of the present invention has an effect of inhibiting the growth of cancer cells by generating photothermal heat due to the effect of absorbing light and dissipating heat.

In addition, the cytotoxicity was assessed by H&E staining of the heart, lung, liver, spleen, kidney and tumor of the mouse illuminated with near infrared light after the injection of the MnCO₃@SFPNPs complex of Example [1-3] above in FIG. 24. According to FIG. 24, no cytotoxicity was observed in the hearts, lungs, livers, spleens, and kidneys of the mouse injected with the PBS, the mouse injected with the PBS followed by near-infrared illumination, the mouse injected with the MnCO₃@SFPNPs, and the mouse injected with the MnCO₃@SFPNPs followed by near-infrared illumination, but the cell death was observed in the tumor of the mouse injected with the MnCO₃@SFPNPs followed by near-infrared illumination. That is, the results as described above suggest that when the tumor cells are injected with the MnCO₃@SFPNPs complex, the near infrared illumination has an effect of selectively inducing the cancer cell death without toxicity to the normal cells.

With the experiments above, as a result, the MnCO₃@SFPNPs complex of the present invention can have low cellular and biological toxicity with eco-friendly fluorescent nanoparticles prepared by using dopamine, which is a biogenic material, and carry metal salts such as manganese carbonate by the catechol functional group. Therefore, it was confirmed that the MnCO₃@SFPNPs complex is a material that enables high-sensitivity diagnosis through the multiplexed imaging of MRI and fluorescence in the specific acidic and low-oxygen environments of cancer cells, and at the same time enables selective photothermal therapy by inhibiting the growth of cancer cells and inducing the cancer cell death due to the capability of absorption of high near-infrared wavelength and release of thermal energy.

1. A complex comprising:
a fluorescent nanoparticle; and
a manganese salt conjugated to a surface of the fluorescent nanoparticle,
wherein fluorescence of the fluorescent nanoparticle is in a state of being quenched due to the conjugation of the manganese salt.

2. The complex of claim 1, wherein the manganese salt is one in which a manganese ion is chelated to the surface of the fluorescent nanoparticle.

3. The complex of claim 2, wherein the manganese ion is chelated by a catechol-derived functional group on the surface of the fluorescent nanoparticle.

4. The complex of claim 3, wherein the catechol-derived functional group is derived from dopamine.

5. The complex of claim 1, wherein the fluorescent nanoparticle is a fluorescent polydopamine nanoparticle.

6. The complex of claim 1, wherein the manganese salt comprises manganese carbonate (MnCO₃), manganese monoxide (MnO), manganese sulfate (MnSO₄), manganese chloride (MnCl₂), manganese nitrate (Mn(NO₃)₂, manganese acetate ((CH₃COO))₂Mn), or a mixture thereof.

7. The complex of claim 1, wherein the fluorescent nanoparticle has a size (D₅₀) of 10 to 300 nm.

8. The complex of claim 1, wherein the fluorescent nanoparticle and the manganese salt are included in a content ratio comprising a manganese ion (Mn²⁺) of 1 mM to 300 mM, based on 1 mg/mL of the fluorescent nanoparticle in an aqueous solution.

9. The complex of claim 1, wherein the complex recovers the fluorescence under an acidic condition.

10. The complex of claim 1, wherein the complex releases thermal energy by light illumination.

11. A composition for bioimaging comprising the complex according to claim 1.

12. A composition for diagnosis of cancer comprising the complex according to claim 1.

13. A composition for prevention or treatment of cancer comprising the complex according to claim 1.

14. The composition of claim 13, wherein the composition releases thermal energy by light illumination.

15. A method for preventing or treating cancer, comprising administering to a subject in need thereof a composition comprising the complex according to claim 1.
